(19) United States
(12) Patent Application Publication
(10) Pub. No.: US 2017/0232050 A1
(43) Pub. Date: Aug. 17, 2017

(54) HERBAL FORMULATION FOR TREATING CHRONIC FATIGUE SYNDROME

(71) Applicant: David ELLERY, Fort Saskatchewan (CA)

(72) Inventor: David ELLERY, Fort Saskatchewan (CA)

(21) Appl. No.: 15/045,130

(22) Filed: Feb. 16, 2016

(51) Int. Cl. A61K 36/45; A61K 36/64; A61K 36/73; A61K 36/16; A61K 36/185; A61K 9/00; A61K 36/71

(52) U.S. Cl. CPC A61K 36/45; A61K 9/0053; A61K 36/64; A61K 36/71; A61K 36/16; A61K 36/185; A61K 36/73

(57) ABSTRACT

In one aspect there is provided a liquid herbal composition for relieving some or all of the symptoms of Seasonal Affective Disorder (SAD) and/or Chronic Fatigue Syndrome (CFS) or Myalgic Encephalomyelitis (ME). The liquid herbal composition preferably comprises therapeutically effective amounts of hydroalcoholic extracts of Bilberry, Eyebright, Goldenseal root, Ginkgo leaf, Passion flower and Raspberry leaf. The liquid herbal composition is preferably orally administered on a daily basis.

### FIELD OF THE INVENTION

The field of present invention relates generally to herbal composition. More particularly, this invention relates to an herbal composition capable of reducing or alleviating the debilitating symptoms of Seasonal Affective Disorder (SAD) and Chronic Fatigue Syndrome (CFS) or Myalgic Encephalomyelitis (ME) in animals, particularly humans. The present invention further relates to methods of using such herbal compositions to reduce or alleviate the symptoms of SAD and CFS/ME in humans.

### BACKGROUND OF THE INVENTION

The background information discussed below is presented to better illustrate the novelty and usefulness of the present invention. This background information is not admitted prior art.

Myalgic Encephalomyelitis (ME) is a severe chronic illness that is more commonly known as Chronic Fatigue Syndrome (CFS). A person with ME/CFS has a number of serious symptoms other than chronic fatigue. The illness affects the brain, central nervous system, heart, muscles and endocrine system. It is considered a neurological, endocrine and an immune system disorder. Some ME/CFS symptoms are similar to the illness known as Fibromyalgia. Seasonal affective disorder (SAD), also known as winter depression, winter blues, summer depression, summertime sadness, or seasonal depression, is a mood disorder subset in which people, who have normal mental health throughout most of the year, experience depressive symptoms in the winter or summer. There are studies which suggest that all of these illnesses are related with common biological underpinnings (see, for example, Juruena, Mario Francisco, & Cleare, Anthony James. (2007). Overlap between atypical depression, seasonal affective disorder and chronic fatigue syndrome. Revista Brasileira de Psiquiatria, 29(Suppl. 1), s19-s26).

Light therapy is used to treat seasonal affective disorder (SAD). The Mayo Clinic states that light therapy is a proven treatment for SAD (see: http://www.mayoclinic.org/tests-procedures/light-therapy/basics/definition/prc-20009617). Further, it has been found that fatigue, excessive daytime sleepiness, and health-related quality of life improve in a similar way as depressed mood (SAD) following treatment with bright light (see, for example: Cecilia Rastad, Jan Ulfberg, and Per Lindberg, “Improvement in Fatigue, Sleepiness, and Health-Related Quality of Life with Bright Light Treatment in Persons with Seasonal Affective Disorder and Subsyndromal SAD,” Depression Research and Treatment, vol. 2011, Article ID 543906, 10 pages, 2011. doi: 10.1155/2011/543906).

However, light therapy may not be suitable for all suffers of SAD and CFS/ME. Light therapy equipment, such as light boxes, can be expensive. Additionally, light therapy typically requires exposure to bright light during daily sessions (e.g. 30 minutes in the morning). This may be undesirably for those individuals with hectic or varied schedules.

Several herbs have been found to increase an individual's sensitivity to light.

For example, Bilberry (Vaccinium myrtillus L.) is used for improving eyesight, including night vision (therefore improving sensitivity to light perception). Bilberry is also used for treating eye conditions such as cataracts and disorders of the retina. Some people use bilberry for conditions of the heart and blood vessels including hardening of the arteries (atherosclerosis), varicose veins, decreased blood flow in the veins, and chest pain. Bilberry Fruit was frequently taken during World War II by British pilots to enhance their night vision before flights.

Bilberry contains anthocyanosides. Plant anthocyanosides have been found to increase the production of rhodopsin, a pigment found in the retina that is involved in the formation of photoreceptor cells. Published data also shows that anthocyanins in whole extract or pure form have antioxidant activity in vitro, often higher than other natural antioxidants. There is evidence from epidemiological studies that high dietary intake of foods rich in flavonoids, including anthocyanins is associated with low prevalence of some diseases such as cancer and cardiovascular disease.

Eyebright (Euphrasia sp., e.g. Euphrasia officinalis) is famous for its use in treating eye related problems, as well as slowing down progressive vision weakness due to age. The whole foliage (stem, leaves, and flowers) of Eyebright is edible and can be used in herbal medicine. The herb's name comes from its common use as an alternative treatment for eyestrain and eye related ailments. The herb is also used to relieve inflammation caused by colds, coughs, sinus infections, sore throats and hay fever. According to researchers, Eyebright herb contains many chemical, organic and nutritional properties that may not only aid in treating eye problems but also may help with a number of other health conditions. It contains vitamins B, C, E, beta-carotene, alkaloids, antioxidants such as caffeic acids, ferulic acids and flavonoids, iridoid glycosides, defensive compounds, tannins and volatile oils.

Goldenseal root (Hydrastis sp. e.g. Hydrastis canadensis L.) was used medicinally by American Indians of the Cherokee, Catawba, Iroquois, and Kickapoo tribes as an insect repellent, a diuretic, a stimulant, and a wash for sore or inflamed eyes. Golden seal has been included in cold and flu preparations for its ability to suppress mucus. Golden seal contains a compound called berberine that kills many types of bacteria in test tubes, including the ones that cause diarrhea (E coli). Berberine also kills a wide range of other types of germs in test tubes, such as those that cause candida (yeast) infections and parasites such as tapeworms and Giardia. Berberine may also activate white blood cells, making them better at fighting infection and strengthening the immune system. Berberine is sometimes used as an antibiotic, and has been studied to treat H. pylori infection (the bacterium that causes ulcers). It is sometimes recommended to treat urinary tract infections (UTIs). Berberine may also be useful in heart failure. One of the precautions given with regard to the use of golden seal root is that it may also cause an increased sensitivity to sunlight.

Ginkgo leaf (Ginkgo biloba) is an herb that the Chinese have medicinally used for centuries, but many of the modern applications come from the research of German scientists. In fact, Ginkgo is a prescription herb in Germany. Ginkgo extract has proven benefits particularly to elderly persons. This ancient herb acts to enhance oxygen utilization and thus improves memory, concentration, and other mental faculties. The herbal extract has also been shown to significantly improve long-distance vision and may reverse damage to the retina of the eye. It increases blood flow to the brain, the uptake of glucose by brain cells, and has been said to improve the transmission of nerve signals.

Most evidence suggests that Ginkgo can slightly improve memory, speed of thinking, and attention in healthy adults. Some evidence shows that taking Ginkgo leaf extract by mouth modestly improves symptoms of Alzheimer's, vascular, or mixed dementias as well as vision problems in people with diabetes. There is further evidence that taking Ginkgo leaf extract by mouth can improve color vision in people with retinas that have been damaged by diabetes as well as vision loss (glaucoma). Taking Ginkgo leaf extract by mouth seems to improve pre-existing damage to the visual field in people with normal tension glaucoma.

Ginkgo biloba increases metabolism efficiency, regulates neurotransmitters, and boosts oxygen levels in the brain. Studies have confirmed that Ginkgo increases blood flow to the retina, and can slow retinal deterioration resulting in an increase of visual acuity.

The medical benefits of Ginkgo biloba extract are attributed primarily to two groups of active constituents: the Ginkgo flavone glycosides and the terpene lactones. Ginkgo flavone glycosides are primarily responsible for Ginkgo's antioxidant activity and may mildly inhibit platelet aggregation (stickiness). These two actions may help Ginkgo prevent circulatory diseases, such as atherosclerosis, and support the brain and central nervous system. In addition to the cardiovascular system, Ginkgo's antioxidant action may also extend to the brain and retina of the eye. Preliminary trials have suggested potential benefit for people with macular degeneration and diabetic retinopathy.

Passiflora, known also as the passion flowers or passion vines, is a genus of about 500 species of flowering plants, the namesakes of the family Passifloraceae. Chemical analysis on passion flower (Passiflora incarnate L.) indicates it contains three main groups of active chemicals: alkaloids, glycosides and flavonoids. Interestingly, when the glycosides and flavonoids are isolated and tested individually they have demonstrated the opposite effects for which the plant is commonly used for. Only when the two groups of chemicals are combined as a whole herb, do researchers observe the plant's sedative effect. Passion flower also contains naturally occurring serotonin as well as a chemical called maltol which has documented sedative effects (and which might explain the natural calming properties of passionflower). Serotonin is popularly thought to be a contributor to feelings of well-being and happiness.

Passionflower is usually used for calming an excited nervous system. Internet and book research indicates that ingesting passionflower can increase levels of the sleep-friendly neurotransmitter GABA. Passionflower blocks the monoamine oxidase digestive enzyme and as a result can have a positive effect on dopamine, norepinephrine, and serotonin. Passionflower also contains naturally occurring serotonin as well as a chemical called maltol which has documented sedative effects (and which might explain the natural calming properties of passionflower). A group of harmane alkaloids in passionflower have demonstrated antispasmodic activity and the ability to lower blood pressure. Furthermore, passion flower is widely used in the United Kingdom for treating anxiety, eye problems, headaches, hypertension, hysteria, insomnia, muscle problems, nerve pain, nerve weakness, pain, restlessness, spasms, and as a relaxant and tranquilizer.

Raspberry leaf (Rubus idaeus) is sometimes called the “woman's herb”. Generally, between 75 to 80% of all sufferers of SAD and ME are women. Red raspberry leaf tea is also incredibly high in valuable vitamins such as vitamin A, B, C, and E, and has an impressive mineral content, offering constituents such as potassium, phosphorus, calcium, and more.

Herbal compounds and phytochemicals can have complementary, synergetic and overlapping mechanisms of action on the human body, including antioxidant effects, alteration of biotransformation enzyme activities, anti-inflammatory effects, stimulation of the immune system, reduction of platelet aggregation, modulation of cholesterol synthesis, hormone metabolism, reduction of blood pressure, and anti-bacterial and antiviral effects.

Therefore, what is desired is a herbal formulation which can reduce or alleviate the symptoms of SAD and CFS/ME, as well as provide antioxidants, including anthocyanins, and various vitamins and which may also have anti-bacterial and antiviral effects, anti-anxiety properties and which can improve memory, concentration, and other mental faculties.

### DETAILED DESCRIPTION OF THE PREFERRED EMBODIMENTS

The following description is of preferred embodiments by way of example only and without limitation to the combination of features necessary for carrying the invention into effect.

The present disclosure is based on the discovery that a combination of certain herbs, blended in appropriate proportions, can reduce or alleviate the symptoms of Seasonal Affective Disorder (SAD) and Chronic Fatigue Syndrome (CFS) or Myalgic Encephalomyelitis (ME) in humans. Preferably, the combination of herbs is provided as an orally administered liquid herbal composition of the hydroalcoholic extracts of:

(A) Bilberry (Vaccinium myrtillus L.) leaves;

(B) Eyebright (Euphrasia officinalis) leaves;

(C) Goldenseal (Hydrastis canadensis L.) root;

(D) Ginkgo (Ginkgo biloba) leaves;

(E) Passion flower (Passiflora incarnate L.) leaves; and

(F) Raspberry (Rubus idaeus) leaves.

This herbal composition will contain “therapeutically effective amounts” of the herbal extracts recited above. As used herein with respect to each of the herbal extracts used in the composition of this invention, the term “therapeutically effective amount” refers to that amount of the hydroalcoholic extract which will contribute to reducing SAD and ME symptoms.

In a first embodiment of the invention, the liquid herbal composition preferably contains hydroalcoholic extracts of the following herbs in the following proportions:

(A) 35 ml of Bilberry (Vaccinium myrtillus L.) leaves;

(B) 15 ml of Eyebright (Euphrasia officinalis) leaves;

(C) 5 ml of Goldenseal (Hydrastis canadensis L.) root;

(D) 15 ml of Ginkgo (Ginkgo biloba) leaves;

(E) 20 ml of Passion flower (Passiflora incarnate L.) leaves; and

(F) 10 ml of Raspberry (Rubus idaeus) leaves.

This first embodiment of the liquid herbal composition may be prepared by combining the above-noted amounts of herbal hydroalcoholic extracts into one liquid composition (i.e. resulting in a total amount of 100 ml). Or it may be prepared by combining the above-noted herbal hydroalcoholic extracts in the following proportions:

(A) 35% by volume of Bilberry (Vaccinium myrtillus L.) leaves hydroalcoholic extract;

(B) 15% by volume of Eyebright (Euphrasia officinalis) leaves hydroalcoholic extract;

(C) 5% by volume of Goldenseal (Hydrastis canadensis L.) root hydroalcoholic extract;

(D) 15% by volume of Ginkgo (Ginkgo biloba) leaves hydroalcoholic extract;

(E) 20% by volume of Passion flower (Passiflora incarnate L.) leaves hydroalcoholic extract; and

(F) 10% by volume of Raspberry (Rubus idaeus) leaves hydroalcoholic extract.

Hydroalcoholic extracts of the above-noted herbal components may be created in a conventional manner, i.e. by placing the dried herbal components in a container and using water and ethanol as the solvents and wherein, preferably, the ethanol percentage is at least 25-60%. For example, the herbs may be put in a container containing a spirit of 40% or more ethanol, such as 80 proof vodka, and wherein the container is then left to stand for at least 2 to 3 weeks; preferably for at least 1 month. The container may also be shaken occasionally in order to maximize extraction.

For example, one can prepare this first embodiment from the dried herbal components of the above-noted herbs using a 1:1 ratio of herb to water-ethanol mixture and using the following weights of dried herbal components:

(A) 35 mg of Bilberry (Vaccinium myrtillus L.) leaves;

(B) 15 mg of Eyebright (Euphrasia officinalis) leaves;

(C) 5 mg of Goldenseal (Hydrastis canadensis L.) root;

(D) 15 mg of Ginkgo (Ginkgo biloba) leaves;

(E) 20 mg of Passion flower (Passiflora incarnate L.) leaves; and

(F) 10 mg of Raspberry (Rubus idaeus) leaves.

In short, the first embodiment of the liquid herbal composition is a hydroalcoholic extract of the following herbal components: Bilberry (Vaccinium myrtillus L.) leaves: Eyebright (Euphrasia officinalis) leaves: Goldenseal (Hydrastis canadensis L.) root: Ginkgo (Ginkgo biloba) leaves: Passion flower (Passiflora incarnate L.) leaves: Raspberry (Rubus idaeus) leaves, in the following ratio: 7:3:1:3:4:2.

A suitable oral dosage of this first embodiment of the liquid herbal composition is between 10 to 30 drops (0.5 to 1.5 ml) taken 1 to 2 times daily either directly or with water. Advantageously, this first embodiment of the liquid herbal composition also provides antioxidants, including anthocyanins, and various vitamins and minerals. The inventor has found that within about 1 to 3 days of such oral administration of this first embodiment of the liquid herbal composition, many symptoms of Seasonal Affective Disorder (SAD) and Chronic Fatigue Syndrome (CFS) or Myalgic Encephalomyelitis (ME) are reduced or completely alleviated. The inventor has further found that, once such oral administration of this first embodiment of the liquid herbal composition ceased, that some of the symptoms of Seasonal Affective Disorder (SAD) and Chronic Fatigue Syndrome (CFS) or Myalgic Encephalomyelitis (ME) would typically return within about 4 days of such cessation, and most of all of the symptoms will have returned within about 10 days of such cessation.

The inventor has further found that when a daily dose greater than 30 drops (5 ml) was taken (e.g. two tablespoons), a significant sensitivity to light developed in the person taking such greater does of the first embodiment of the liquid herbal composition. The inventor therefore believes that this first embodiment of the liquid herbal composition, when taken orally as described above, acts to increase a person's sensitivity to light. This increase in light sensitivity then results in an overall lower need for light by such person (e.g. during winter months or at higher latitudes), which in turn appears to lower or fully alleviated symptoms of Seasonal Affective Disorder (SAD) and Chronic Fatigue Syndrome (CFS) or Myalgic Encephalomyelitis (ME).

A second embodiment of the liquid herbal composition can be prepare from the dried herbal components of the above-noted herbs using a 1:1 ratio of herb to water-ethanol mixture and using the following weights of dried herbal components:

(A) 900 mg of Bilberry (Vaccinium myrtillus L.) fruit;

(B) 535 mg of Eyebright (Euphrasia officinalis) whole plant;

(C) 300 mg of Goldenseal (Hydrastis canadensis L.) root and rhizome;

(D) 2000 mg of Ginkgo (Ginkgo biloba) leaves;

(E) 125 mg of Passion flower (Passiflora incarnate L.) herb top; and

(F) 155 mg of Raspberry (Rubus idaeus) leaves.

In short, the second embodiment of the liquid herbal composition is a hydroalcoholic extract of the following herbal components: Bilberry (Vaccinium myrtillus L.) fruit: Eyebright (Euphrasia officinalis) whole plant: Goldenseal (Hydrastis canadensis L.) root and rhizome: Ginkgo (Ginkgo biloba) leaves: Passion flower (Passiflora incarnate L.) herb top: Raspberry (Rubus idaeus) leaves; in the following ratio: 7.2:4.28:2.4:16:1:1.24.

The second embodiment of the liquid herbal composition may be prepared by combining the above-noted herbal hydroalcoholic extracts in the following proportions:

(A) at least 22.4% by volume of Bilberry (Vaccinium myrtillus L.) fruit hydroalcoholic extract;

(B) at least 13.3% by volume of Eyebright (Euphrasia officinalis) whole plant hydroalcoholic extract;

(C) at least 7.4% by volume of Goldenseal (Hydrastis canadensis L.) root and rhizome hydroalcoholic extract;

(D) at least 49.8% by volume of Ginkgo (Ginkgo biloba) leaves hydroalcoholic extract;

(E) at least 3.1% by volume of Passion flower (Passiflora incarnate L.) herb top hydroalcoholic extract; and

(F) at least 3.8% by volume of Raspberry (Rubus idaeus) leaves hydroalcoholic extract.

Preferably, the Ginkgo extract contains at least 24% flavonoid glycosides and 6% terpene lactones. Advantageously, this second embodiment provides a high proportion of antioxidants, in addition to providing and various vitamins and minerals and alleviating symptoms of Seasonal Affective Disorder (SAD) and Chronic Fatigue Syndrome (CFS) or Myalgic Encephalomyelitis (ME).

A suitable oral dosage of this second embodiment of the liquid herbal composition is 10 drops (0.5 ml) taken 2 times daily either directly or with water or food.

Advantageously, each of the herbal components in the embodiments of the invention are generally recognized as herbs which can be safely taken over a long term time frame. As such, when consumed on a daily basis as described above, the embodiments the liquid herbal composition of the invention can provide continuous relief of some or all of the symptoms of Seasonal Affective Disorder (SAD) and Chronic Fatigue Syndrome (CFS) or Myalgic Encephalomyelitis (ME) for many years. More advantageously, use of the liquid herbal composition of the invention by a sufferer of Seasonal Affective Disorder (SAD) and/or Chronic Fatigue Syndrome (CFS) or Myalgic Encephalomyelitis (ME) results in such user being able to reduce or eliminate their previous light therapy.

Those of ordinary skill in the art will appreciate that various modifications to the invention as described herein will be possible without falling outside the scope of the invention. In the claims, the word “comprising” is used in its inclusive sense and does not exclude other elements being present. The indefinite article “a” before a claim feature does not exclude more than one of the features being present.

1. An orally administered liquid herbal composition for relieving some or all of the symptoms of Seasonal Affective Disorder (SAD) or Chronic Fatigue Syndrome (CFS) or Myalgic Encephalomyelitis (ME), comprising:
therapeutically effective amounts of hydroalcoholic extracts of Bilberry;
therapeutically effective amounts of hydroalcoholic extracts of Eyebright;
therapeutically effective amounts of hydroalcoholic extracts of Goldenseal root;
therapeutically effective amounts of hydroalcoholic extracts of Ginkgo leaf;
therapeutically effective amounts of hydroalcoholic extracts of Passion flower; and
therapeutically effective amounts of hydroalcoholic extracts of Raspberry leaf.

2. The liquid herbal composition of claim 1 wherein 22.4% to 35% of the volume is comprised of the hydroalcoholic extract of Bilberry.

3. The liquid herbal composition of claim 1 wherein 13.3% to 15% of the volume is comprised of the hydroalcoholic extract of Eyebright.

4. The liquid herbal composition of claim 1 wherein 5% to 7.4% of the volume is comprised of the hydroalcoholic extract of Goldenseal root.

5. The liquid herbal composition of claim 1 wherein 15% to 49.8% of the volume is comprised of the hydroalcoholic extract of Ginkgo leaf

6. The liquid herbal composition of claim 1 wherein 3.1% to 20% of the volume is comprised of the hydroalcoholic extract of Passion flower.

7. The liquid herbal composition of claim 1 wherein 3.8% to 10% of the volume is comprised of the hydroalcoholic extract of Raspberry leaf.

8. The liquid herbal composition of claim 1 comprising:
at least 22.4% by volume of the hydroalcoholic extract of Bilberry;
at least 13.3% by volume of the hydroalcoholic extract of Eyebright;
at least 5% by volume of the hydroalcoholic extract of Goldenseal root;
at least 15% by volume of the hydroalcoholic extract of Ginkgo leaf;
at least 3.1% by volume of the hydroalcoholic extract of Passion flower; and
at least 3.8% by volume of the hydroalcoholic extract of Raspberry leaf.

9. The use of a liquid herbal composition to relieve some or all of the symptoms of Seasonal Affective Disorder (SAD) or Chronic Fatigue Syndrome (CFS) or Myalgic Encephalomyelitis (ME), said liquid herbal composition comprising:
therapeutically effective amounts of hydroalcoholic extracts of Bilberry;
therapeutically effective amounts of hydroalcoholic extracts of Eyebright;
therapeutically effective amounts of hydroalcoholic extracts of Goldenseal root;
therapeutically effective amounts of hydroalcoholic extracts of Ginkgo leaf;
therapeutically effective amounts of hydroalcoholic extracts of Passion flower; and
therapeutically effective amounts of hydroalcoholic extracts of Raspberry leaf.

10. The use of the liquid herbal composition of claim 9 to relieve some or all of the symptoms of Seasonal Affective Disorder (SAD) or Chronic Fatigue Syndrome (CFS) or Myalgic Encephalomyelitis (ME), wherein the liquid herbal composition comprises:
at least 22.4% by volume of the hydroalcoholic extract of Bilberry;
at least 13.3% by volume of the hydroalcoholic extract of Eyebright;
at least 5% by volume of the hydroalcoholic extract of Goldenseal root;
at least 15% by volume of the hydroalcoholic extract of Ginkgo leaf;
at least 3.1% by volume of the hydroalcoholic extract of Passion flower; and
at least 3.8% by volume of the hydroalcoholic extract of Raspberry leaf.

11. The use of the liquid herbal composition of claim 9 to relieve some or all of the symptoms of Seasonal Affective Disorder (SAD) or Chronic Fatigue Syndrome (CFS) or Myalgic Encephalomyelitis (ME), wherein 22.4% to 35% of the volume of the liquid herbal composition is comprised of the hydroalcoholic extract of Bilberry

12. The use of the liquid herbal composition of claim 9 to relieve some or all of the symptoms of Seasonal Affective Disorder (SAD) or Chronic Fatigue Syndrome (CFS) or Myalgic Encephalomyelitis (ME), wherein 13.3% to 15% of the volume of the liquid herbal composition is comprised of the hydroalcoholic extract of Eyebright (New) The use of the liquid herbal composition of claim 9 to relieve some or all of the symptoms of Seasonal Affective Disorder (SAD) or Chronic Fatigue Syndrome (CFS) or Myalgic Encephalomyelitis (ME), wherein 5% to 7.4% of the volume of the liquid herbal composition is comprised of the hydroalcoholic extract of Goldenseal root.

14. The use of the liquid herbal composition of claim 9 to relieve some or all of the symptoms of Seasonal Affective Disorder (SAD) or Chronic Fatigue Syndrome (CFS) or Myalgic Encephalomyelitis (ME), wherein 15% to 49.8% of the volume of the liquid herbal composition is comprised of the hydroalcoholic extract of Ginkgo leaf.

15. The use of the liquid herbal composition of claim 9 to relieve some or all of the symptoms of Seasonal Affective Disorder (SAD) or Chronic Fatigue Syndrome (CFS) or Myalgic Encephalomyelitis (ME), wherein 3.1% to 20% of the volume of the liquid herbal composition is comprised of the hydroalcoholic extract of Passion flower

16. The use of the liquid herbal composition of claim 9 to relieve some or all of the symptoms of Seasonal Affective Disorder (SAD) or Chronic Fatigue Syndrome (CFS) or Myalgic Encephalomyelitis (ME), wherein 3.8% to 10% of the volume of the liquid herbal composition is comprised of the hydroalcoholic extract of Raspberry leaf.

17. A method of treating a symptom of Seasonal Affective Disorder (SAD) or Chronic Fatigue Syndrome (CFS) or Myalgic Encephalomyelitis (ME), comprising administering to a subject suffering from Seasonal Affective Disorder (SAD) or Chronic Fatigue Syndrome (CFS) or Myalgic Encephalomyelitis (ME) an amount of an herbal composition sufficient to treat the symptom of Seasonal Affective Disorder (SAD) or Chronic Fatigue Syndrome (CFS) or Myalgic Encephalomyelitis (ME), wherein said herbal composition comprises:
therapeutically effective amounts of hydroalcoholic extracts of Bilberry;
therapeutically effective amounts of hydroalcoholic extracts of Eyebright;
therapeutically effective amounts of hydroalcoholic extracts of Goldenseal root;
therapeutically effective amounts of hydroalcoholic extracts of Ginkgo leaf;
therapeutically effective amounts of hydroalcoholic extracts of Passion flower; and
therapeutically effective amounts of hydroalcoholic extracts of Raspberry leaf.

18. The method of claim 17, wherein the symptom is one of: fatigue, depression or excessive daytime sleepiness.

19. The liquid herbal composition of claim 1, wherein at least one of the symptoms is fatigue, depression or excessive daytime sleepiness.

20. The use of the liquid herbal composition of claim 9, wherein at least one of the symptoms is fatigue, depression or excessive daytime sleepiness.
